(19) United States
(12) Patent Application Publication
(10) Pub. No.: US 2025/0186636 A1
(43) Pub. Date: Jun. 12, 2025

(54) PROCESS FOR THE PREPARATION OF STERILE PRODUCTS

(71) Applicant: CMED Aesthetics S.P.A., Pisa (IT)

(72) Inventors: Carlo MICHELINI, Livorno (IT); Andrea MALVENTI, San Giuliano Terme (PI) (IT); Laura ZAPPOLINI, Cascina (PI) (IT); Andrea DALCANTO, Collesalvetti-Vicarello (LI) (IT)

(73) Assignee: CMED Aesthetics S.P.A., Pisa (IT)

(21) Appl. No.: 18/843,845

(22) PCT Filed: Mar. 3, 2023
(86) PCT No.: PCT/EP2023/055427

§ 371 Date: Sep. 4, 2024

(30) Foreign Application Priority Data
Mar. 10, 2022 (IT) 102022000004613

(51) Int. Cl. A61L 2/02; A61K 8/73; A61L 2/00; A61Q 19/08; B01D 63/14; B01D 69/02; B01D 69/10; B01D 71/68

(52) U.S. Cl. CPC A61L 2/022; A61K 8/735; A61L 2/0017; A61Q 19/08; B01D 63/14; B01D 69/02; B01D 69/10; B01D 71/68; A61K 2800/91; A61L 2202/21; B01D 2313/44; B01D 2325/02834

(57) ABSTRACT

A process for the preparation of sterile products comprising thermolabile and/or bioactive raw materials. The process comprises a step of sterilizing the products by filtration. Preferably, the sterile products obtained by the process are for cosmetic use, more preferably for use in cosmetic mesotherapy, such as anti-aging mesotherapy, or for medical use in aesthetic medicine or dermatology, preferably as injectable products. The process of the invention is particularly preferred for the preparation of sterile hyaluronic acids, or derivatives thereof.

### BACKGROUND OF THE INVENTION

The present invention relates to a process for the preparation of sterile products comprising thermolabile and/or bioactive raw materials, such as vitamins, peptides, polypeptides, sodium deoxycholate, polymers, such as hyaluronic acid or derivatives thereof, other biopolymers with structural protein function, such as collagen, keratin, elastin, functional proteins, such as enzymes and hormones, functional polysaccharides, such as cellulose, cellulosic polymers and derivatives thereof, chitin, among others.

Such a process comprises a phase of sterilization by filtration of said products.

Preferably, said sterile products are for cosmetic use, more preferably for use in cosmetic mesotherapy, such as anti-aging mesotherapy, or for medical use, in particular in aesthetic medicine or dermatology, preferably as injectable products. Particularly preferred active polymers included in said sterile products are hyaluronic acid and derivatives thereof.

### BACKGROUND ART

Mesotherapy is a technique of administering active substances intraepidermally, superficial and deep intradermally, and subcutaneously or hypodermically.

The advantage of this technique consists in being able to use reduced doses of bioactive raw materials, which diffuse in the tissues underlying the inoculation and persist longer as compared to the intramuscular route of administration, with advantages such as the prolonged effect over time, the reduced involvement of other organs and the reduction of the risk of adverse events or side effects.

The main applications of mesotherapy comprise medical therapies, such as the therapy of pain, trauma, arterial disease, phlebolymphedema, dermatological therapies, as well as cosmetic treatments.

In the cosmetic field, anti-aging mesotherapy (MAE) is widely used, which consists in applying very small amounts of hyaluronic acid (AI) associated or not with a cocktail of further active substances or moisturizers at the epidermal level. In such a context, MAE is also called biorejuvenation, biorivitalization or mesolift.

The purpose thereof is to increase the biosynthesis capacity, by the fibroblast, of neocollagen, elastin and AI, which has, as a result, an increase in the firmness, brightness and hydration of the skin.

In general, products comprising active raw materials and in particular polymers are employed in the cosmetic field, preferably administered by mesotherapy techniques.

The term “active” or “bioactive” in reference to a raw material, such as a polymer, indicates that said raw material possesses a biological function and/or activity, including the mechanical activities carried out by structural polymers, in particular proteins, in the cells or in the extra-cellular matrix of an organism.

“Thermolabile” raw materials means raw materials the chemical-physical features of which and/or the functionality of which are altered by heat; in particular, “thermolabile” raw materials according to the purposes of the present invention, are raw materials the chemical-physical features of which and/or the functionality of which are altered by temperatures greater than or equal to 120° C., such as the temperatures typically reached during autoclave sterilization treatments.

The price of raw materials used also in cosmetics and in particular in MAE is very high. Therefore, it is highly desirable that the preparation of products comprising them does not result in their deterioration.

Moreover, the very effectiveness of such raw materials depends on the integrity of the functional and structural-rheological features thereof. For example, the effectiveness of hyaluronic acid or derivatives thereof, for all medical or cosmetic applications, is closely linked to the integrity of the molecules forming it and to the rheological properties of the product.

However, the production processes of sterile products require a sterilization procedure, which can result in variations in the structure and functionality of the raw materials included in the product with respect to the original ones.

Typically, the sterilization can be performed with one or more of the methods described below. The procedure chosen must always be validated, with respect to both the effectiveness thereof and the integrity of the product, comprising the container thereof or the packaging thereof, at the end of the sterilization. Furthermore, for all the sterilization methods, the critical process parameters are controlled so as to confirm that the previously determined required conditions are reached in the batch during the entire sterilization process.

For example, in terminal sterilization, i.e., the sterilization of the packaged product, it is of paramount importance to consider the non-uniformity of the physical and, where relevant, chemical conditions inside the sterilization chamber, determining the location inside the sterilization chamber which is least accessible to the sterilizing agent, the minimum degree of lethality produced by a sterilization cycle and the reproducibility of the cycle to ensure that all the loads receive the specified treatment. Other combinations of time and temperature can be used for all the processes as long as it has been satisfactorily demonstrated that the chosen process obtains an adequate and reproducible lethality level when routinely carried out within the established tolerance thresholds. The procedures and precautions employed are such as to confer an LAS of 10⁻⁶ or better.

In sterilization by saturated pressurized steam (autoclave heating), preferably for aqueous preparations, the reference conditions are heating at a minimum temperature of 121° C. for 15 min. The temperature is usually measured by thermosensors inserted in the containers together with other elements positioned in the previously established less hot part of the loading chamber. The parameters of each cycle are appropriately recorded, for example, through the temperature/time graph or through any other suitable means. The biological evaluation of the sterilized product is carried out using an appropriate biological indicator. In the event of dry heat sterilization, the reference conditions are a minimum of 160° C. for at least 2 h. Dry heat sterilization is performed in forced air ventilation stoves or other similar equipment designed for this purpose. The sterilizing apparatus is loaded so that a uniform temperature is reached throughout the load. The temperature reached inside the sterilizer during the sterilization process is usually measured by thermosensors inserted in representative containers together with other elements in the previously established less hot part of the sterilizer. For the whole duration of each cycle, the temperature is recorded appropriately. Dry heat at temperatures above 220° C. is often used for the sterilization and depyrogenation of glassware. In this case, the biological indicator can be replaced by demonstrating a 3 log reduction in the amount of heat-resistant endotoxins.

Ionizing radiation sterilization is performed by exposing the product to ionizing radiation in the form of gamma radiation generated by an appropriate radioisotope source (such as cobalt 60) or by a beam of electrons energized by an appropriate electronic accelerator. During the sterilization process, the radiation absorbed by the product is regularly controlled by defined dosimetry procedures which allow a real measurement of the dose received by the product, regardless of the generated radiation rate. The dosimeters are calibrated with respect to a standard source of a reference irradiation system, both at the time they are received from the supplier and at appropriate intervals of not more than one year.

Gas sterilization is used only in the absence of appropriate alternatives. It is of paramount importance that the gas and moisture penetration into the material to be sterilized is ensured and that it is followed by a gas removal process under conditions which have been previously established to ensure that any gas residue or the transformation products thereof in the sterilized product are below the potentially toxic concentration during the use of the product. Where possible, gas concentration, relative humidity, temperature and process duration are measured and recorded. The measurements are made where the sterilization conditions are least likely to be achieved, as determined upon validation. The effectiveness of the process applied to each sterilization load is verified using an appropriate biological indicator.

The raw materials and products that cannot be terminally sterilized can be subjected to a filtration procedure through a filter the efficacy of which has been demonstrated by a sample microbial infection test performed with a suitable test micro-organism, such as a decreased Pseudomonas suspension (ATCC 19146, NCIMB 11091 or CIP 103020). In such a case, at least 107 CFUs are used per cm² of active filtering surface, preparing the suspension in soy-tryptone broth. After passing through the filter the suspension is collected aseptically and incubated under aerobic conditions at 32° C.

These products require special precautions. The production process and the environment are chosen so as to minimize microbial contamination and are regularly subject to appropriate control procedures. The equipment, containers and closures and, where possible, ingredients are subjected to an appropriate sterilization process.

The filtration must also be performed as soon as possible after the preparation of the product and the post-filtration operations must be performed under aseptic conditions.

Typically, filtration occurs through membranes, which retain the bacteria, having pores with a nominal diameter of 0.22 μm or less, or through any other filter having the same bacteria-retaining properties. Appropriate measures are taken to prevent loss of solute by adsorption on the filter and to avoid the release of contaminants from the latter. It is necessary to take into account the microbial contamination before filtration, filter capacity, batch size and filtration duration. The filter must not be used for longer periods than those approved following combined validation of the filter itself and the product to be filtered. The integrity of an assembled sterilizing filter is checked before its use, and confirmed after its use, by tests appropriate to the type of filter used and to the stage of the test in which the verification is performed such as, for example, the bubbling point, the pressure seal or diffusion rate tests. Due to other potential drawbacks of the filtration method compared to other sterilization processes, a pre-filtration is generally performed through a filter which retains bacteria and other particulate contaminants, in the event that a decrease in the sterilizing pre-filtration bioburden cannot be ensured with other processes.

The process of filtration of thermolabile substances in order to sterilize said substances has been known for quite a long time, see e.g. the document Soelkner P et al., “Cartridge Filters”, Filtration in the biopharmaceutical industry, 1988, pages 145-168, which illustrates the common general knowledge in the art. The text describes different kinds of filters: among others, pleated cartridge filters made of sundry materials, particularly filters in polyethersulfone, with pore sizes ranging 0.05 and 5 μm.

Products comprising active polymers such as hyaluronic acid or derivatives thereof, with a thermosensitive content and with a relatively high viscosity, can however cause filterability problems.

“Filterability” means the filtering capacity of a filter in terms of flow and saturation, respectively below which and beyond which the filter is not suitable for the treatment of a product to be sterilized.

Furthermore, the filtration of such products must not result in undesired variations in the filtered product with respect to the pre-filtration product, for both the individual raw materials and the functional and structural features of the filtered product, since the filtered product must maintain the functionalities of the pre-filtration product.

Therefore, the filtered product must maintain the individual raw materials intact and maintain the viscosity of the starting product; moreover, the same pre-filtration product must have features such as not to negatively impact the filterability.

Therefore, it is the object of the present invention to provide a process for the preparation of sterile products comprising thermolabile and/or bioactive raw materials, in particular polymers, which is effective and provides a filtered product which maintains the chemical-physical features and functional efficacy of the product before filtration. Furthermore, it is the object of the present invention to provide a process which allows maintaining a high filterability even in the presence of viscous products.

### BRIEF DESCRIPTION OF THE DRAWINGS

Said object has been achieved by the process of the present invention, the features and advantages of which will become apparent from the following detailed description, the embodiments provided by way of illustrative and non-limiting examples, and the accompanying drawings, in which:

FIG. 1 consists of FIGS. 1A, 1B, and 1C and shows the effect of temperature on the viscosity of a product comprising 1.6 MDa hyaluronic acid at 0.5% w/w and 0.8% w/w sodium chloride in water. FIG. 1A shows the viscosities of the products after different heat and stirring treatments, measured at room temperature (about 20° C.); FIG. 1B shows the viscosities of the products after different heat treatments, measured at treatment temperatures or at temperatures below the previous treatments by 10° C., at most; FIG. 1C groups the results shown in FIGS. 1A and 1B (histograms with the same color or texture in FIGS. 1A, 1B and 1C represent the same heat treatment);

FIG. 2 shows the effect of the same heat treatment and stirring on the viscosity of different products comprising an increasing content of hyaluronic acid according to Example 2.

FIG. 3 shows the variation of the viscosity of two products (D and E of Example 2) in different steps of the process, before and after filtration, with respect to the same unfiltered products, according to Example 4.

FIG. 4 shows the comparison between a sample of MESO HAIR REV01 of Example 7 sterilized by filtration according to the method of the present invention (image on the left) and a sample of the same formulation sterilized in autoclave (image on the right).

FIG. 5 shows the comparison between a sample of MESO WHITE REV01 of Example 7 sterilized by filtration according to the method of the present invention (image on the left) and a sample of the same formulation sterilized in autoclave (image on the right).

FIG. 6 shows a sample of SODIUM DEOXYCHOLATE REV02 from Example 7 after sterilization in autoclave.

FIG. 7 consists of FIGS. 7A, 7B, and 7C, and shows a cartridge filter (1) according to a preferred aspect of the invention; two images of the filter (1) are shown in cross-section in A: on the left the filter is assembled inside a cylindrical container (3) and a central duct (4) of the cartridge is visible, on the right the filter is disassembled from the container (3); in B the filter (1) is shown in side view, extracted from the container (3); in C the layers of the multilayer cartridge filter (1) are shown in cross-section: on the left the detail of the pleating is visible with the upstream external (10a) and internal (10b) pleats; on the right the filter layers are mutually separated, to show the central filtering membrane (11) and the external (12a) and internal (12b) support membranes, on each side of the filtering membrane (11); the dashed arrow indicates the flow direction of the filtered solution, from the outside (a) to the inside (b) the filter.

FIG. 8 consists of FIGS. 8A, 8B, and 8C and shows a cartridge filter (2) according to a particularly preferred aspect of the invention; two images of the filter (2) are shown in cross-section in A: on the left the filter is assembled inside a cylindrical container (3) and a central duct (4) is visible, on the right the filter is disassembled from the container; in B the filter (2) is shown in side view, extracted from the container (3); in C the layers of the multilayer cartridge filter (2) are shown in cross-section: on the left the detail of the pleating is visible with the upstream external (20a) and internal (20b) pleats, on the right the filter layers are mutually separated, to show the filtering membrane (21), the external (22a) and internal (22b) support membranes, on each side of the filtering membrane (21), and the stiff lattices, two outermost (23a and 23′a) and two innermost (23b and 23′b); the dashed arrow indicates the flow direction of the filtered solution, from the outside (a) to the inside (b) of the filter.

FIG. 9 shows all the layers of the filter in FIG. 8: from left to right the second and the first external stiff lattice (23a′ and 23a′), the external support membrane (22a), the filtering membrane (21), the internal support membrane (22b), the first and the second internal stiff lattice (23b and 23b′) are visible; the arrow indicates the flow direction of filtration, from the external surface (a) to the internal surface (b) of the filter.

FIG. 10 shows a diagram of the filter (2) in FIG. 9: the filtering membrane (21), the external (22a) and internal (22b) support membranes and the external (23a, 23′a) and internal (23b, 23′b) stiff lattices are visible; the flow direction of the filtered solution through the filter, from the outside (a) to the inside (b), is indicated with an arrow; the external inter-pleat space (a′) between two external upstream pleats (20a) and the internal inter-pleat space (b′) between two internal upstream pleats (20b) is also indicated.

### DETAILED DESCRIPTION OF THE INVENTION

The present invention relates to a process for the preparation of sterile products, the process comprising in sequence the phases of:
i. providing an aqueous solution comprising a thermolabile and/or bioactive raw material,
ii. pre-treating said solution maintaining it under stirring for 1-6 hours at 40-70° C., preferably at a speed of 100-5.000 rpm (revolutions per minute of the stirring rod);
iii. filtering said aqueous solution through at least one filter, at the same temperature of the pre-treatment phase ii), wherein the flow of said aqueous solution through said filter defines an external surface (a) through which the aqueous solution enters into the filter and an internal surface (b) through which the sterile product exits;
wherein said filter is a pleated filter comprising at least one polyethersulfone (PES) filtering membrane, with pores of size less than or equal to 0.25 μm.

Single- and multi-layer PES filters are typically used for the sterilization of aqueous liquids; the pore sizes vary based on the purpose. Such filters have low affinity for proteins and other molecules which can bind to the membrane, offer a high filtration flow, and are compatible with a wide pH range. Furthermore, they can be washed to remove organic and inorganic material which has been retained by the filter.

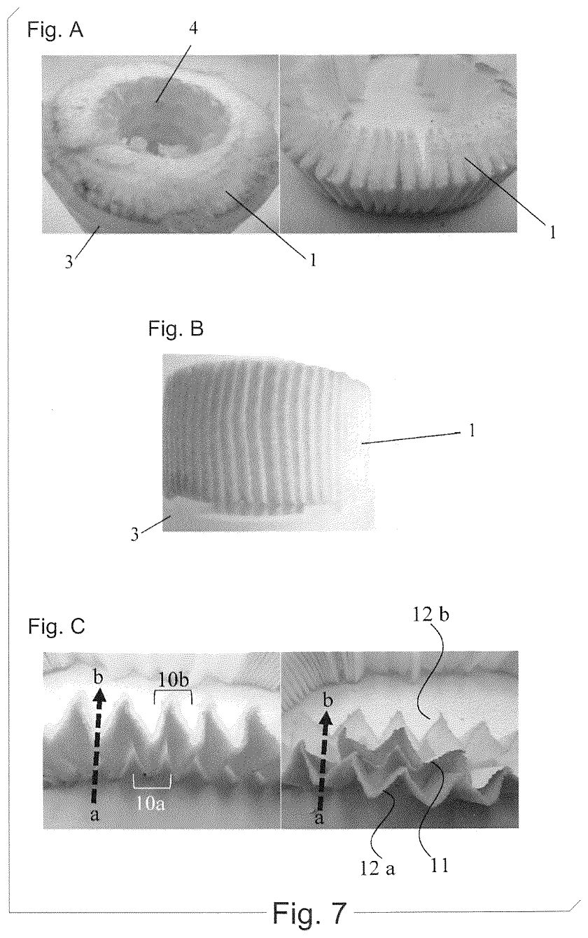

The flow direction through the filter is shown by the orientation of the arrows in the left images in FIGS. 7C, 8C and FIGS. 9 and 10, where the external surface (a) through which the aqueous solution enters the filter and the internal surface (b) through which the sterile product exits are visible.

Preferably, said pleated filter is a multilayer pleated filter.

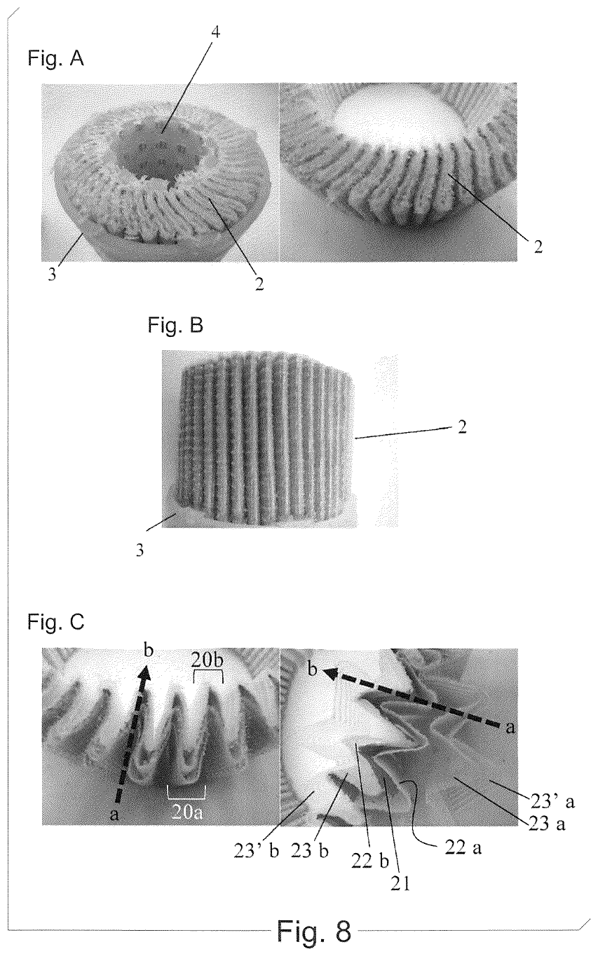

Preferably, said pleated filter is a cartridge filter (1, 2), as shown in FIGS. 7A and 8A, wherein the external surface (a) of the filter faces the internal wall of a container (3) in which the filter is housed, while the internal surface (b) of the filter defines a central duct (4).

“Pleated filter” means a pleated or folded filter.

In preferred embodiments wherein said pleated filter is a cartridge multilayer pleated filter (1, 2), upstream pleats (10a, 20a) alternate on the external surface (a) of the filter, in which the pleat line is on the internal side (b) of the filter, and downstream pleats, wherein the pleat line is on the external side (a) of the filter; said downstream pleats on the external side of the filter correspond to upstream pleats on the internal side of the filter (10b, 20b), as seen in FIGS. 7C and 8C, to the left and in the diagram in FIG. 10.

On the external side of the filter, adjacent upstream pleats define an external inter-pleat space (a′) therebetween; on the internal side of the filter, adjacent upstream pleats define an external inter-pleat space (b′) therebetween, as diagrammed in FIG. 10.

Preferably, said multilayer filters have layers with overlapping and crossed membranes.

Said multilayer pleated filter (1, 2) comprises at least three layers: a central layer consisting of said at least one polyethersulfone filtering membrane (11, 21) in a sandwich between two further layers, consisting of an external support membrane (12a, 22a) on one side of the filtering membrane, and of an internal support membrane (12b, 22b) on the other side of the filtering membrane, as shown in the image on the right in FIG. 7C, said support membranes being suitable for distancing the external upstream pleats (10a, 20a) of the filter apart, defining an inter-pleat space (a′), preferably of 0.1-10 mm, more preferably of 0.5-5 mm, even more preferably of 0.5-2 mm. The distance between two pleats (inter-pleat space) is preferably measured between the vertices of two adjacent pleats.

Preferably said support membranes, too, are made of polyethersulfone, or nylon, cellulose or derivatives thereof.

Preferably said filtering membrane (11, 21) has pores of 0.22 μm; preferably said support membranes (12, 22) have pores with dimensions greater than 0.22 μm, for example pores with dimensions from 0.25 μm to 500 μm, from 0.45 to 500 μm, from 0.65 to 500 μm, even more preferably from 100 to 500 μm.

By advantageously avoiding the pleats from mutually approaching, the support membranes (12, 22) improve the filterability in the process of the invention.

More preferably, the filter further comprises at least one, even more preferably at least two, stiff lattices (23 a and b, 23′a and b) after each support membrane (22a and 22b), as shown in the image to the right in FIG. 8C, said stiff lattices being adapted to form, together with the support membranes, a physical thickness, preferably of 0.5-10 mm, more preferably of 1-5 mm, such that both the external upstream pleats (20a) and the internal upstream pleats (20b) are further expanded with respect to a filter which does not comprise the stiff lattices.

Advantageously, in fact, the stiff lattices are suitable for spacing both the internal pleats (20b), increasing the internal inter-pleat spaces (b′), preferably by at least 500 μm, and the external pleats (20a), increasing the external inter-pleat spaces (a′) by at least 1000 μm, with respect to a filter which does not comprise the stiff lattices, as seen in FIGS. 8A and B and schematized in FIG. 10.

The term “stiff lattice” is intended preferably as a mesh lattice, more preferably 0.5-1.5 mm oval mesh, characterized by a flexibility lower than the flexibility of the filtering and support membranes; thereby said stiff lattices contribute to maintaining the pleats open as well as spaced apart from one another. Materials adapted to form said stiff lattices are polypropylene or high density polyethylene.

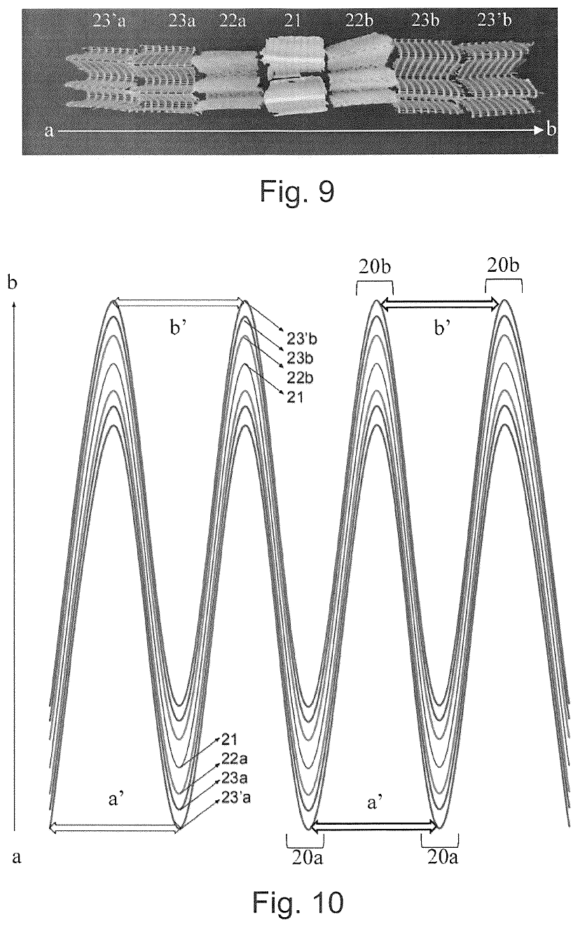

In a particularly preferred aspect the pleated multilayer filter (2) thus comprises, from the external surface (a) to the internal surface (b): a second stiff lattice (23′a) and a first stiff lattice (23a), a first external support membrane (22a), the filtering membrane (21), a second internal support membrane (22b), a first stiff lattice (23b) and a second internal stiff lattice (23′b), as shown in FIGS. 8 and 9. The flow of the aqueous solution during the filtration proceeds from the outside (a) towards the inside (b) of the cartridge (1,2), passing through all the layers of the filter (2), from the outermost to the innermost, as shown in FIG. 9.

More preferably the pleats (20a, 20b) are conical-shaped pleats.

The filters employed in the process of the present invention have a higher efficiency in terms of filterability, from the point of view of both the filtered volume and the low interaction with the raw materials used.

Preferably, the sterile products obtainable with the process of the present invention are products for medical and/or cosmetic use, more preferably for use in cosmetic mesotherapy, such as anti-aging mesotherapy.

Said sterile products preferably comprise as raw material hyaluronic acid or a derivative thereof, and/or a thermolabile raw material.

The term “hyaluronic acid derivative” is meant as comprising:
esters of hyaluronic acid in which part or all of the carboxylic groups are esterified with alcohols of the aliphatic, aromatic, arylaliphatic, cycloaliphatic, heterocyclic series, as also described in EP0216453 B1,
self-cross-linked esters of hyaluronic acid in which part or all of the carboxylic groups are esterified with the alcoholic groups of the same polysaccharide chain or of other chains, as also described in EP0341745 B1,
cross-linked hyaluronic acid compounds in which part or all of the carboxylic groups are esterified with polyalcohols of the aliphatic, aromatic, arylaliphatic, cycloaliphatic, heterocyclic series, generating cross-links by spacer chains, as also described in EP0265116 B1,
hemiesters of succinic acid or heavy metal salts of succinic acid with hyaluronic acid or with partial or total esters of hyaluronic acid, as also described in WO96/357207,
O-sulfated derivatives, as also described in WO95/25751 A1, or N-sulfated derivatives, as also described in WO/1998/045335 A1, and mixtures thereof.

In particularly preferred aspects, said hyaluronic acid or derivative thereof has molecular weight from 500 Da to 3 MDa.

Unless otherwise indicated, the concentrations of one or more ingredients in the aqueous solution or sterile product are referred to herein as concentrations by weight (w/w), based on the total weight of the aqueous solution or sterile product, respectively.

Preferably, the concentration of said hyaluronic acid or a derivative thereof in the aqueous solution is 0.05-10% w/w, more preferably 0.1-10% w/w, even more preferably 1-10% w/w, based on the total weight of the aqueous solution.

Advantageously, in fact, the process of the present invention also allows sterilizing solutions comprising high concentrations of polymers.

Preferably, the viscosity of said aqueous solution comprising the thermolabile and/or bioactive raw material and of said sterile products is between 100 cP and 10,000 cP.

The process of the present invention is particularly advantageous for sterilizing aqueous solutions having viscosity between 100 cP and 10,000 cP, more preferably between 1000 cP and 10,000 cP, obtaining sterile products the viscosity of which at room temperature is substantially unchanged with respect to the viscosity at room temperature of the pre-sterilization solution (±15% at most, preferably ±10% at most, of post-sterilization viscosity variation).

Preferably, phase ii) of maintaining said aqueous solution under stirring at a speed of 100-5,000 rpm, is carried out for 1-4 hours, more preferably for about 2 hours, at a temperature of about 60° C., or overnight at room temperature.

The term “overnight” indicates that certain conditions are maintained for 10-18 hours, typically for about 12 hours.

For the purposes of the present invention, a temperature is “about” equal to a certain value, when it is equal to such a value ±2° C.

“Room temperature” is meant herein as a temperature of 20° C.±2° C.

Without wishing to be bound by theory, it is believed that stirring the solution at the indicated rate and for the indicated period of time followed by filtration results in a stabilization of the formulation favoring the alignment of the polymer chains of hyaluronic acid or derivative thereof, while maintaining the viscosity of the solution at the desired level and constant over time.

Preferably the aqueous solution comprises salts, more preferably sodium chloride (NaCl), in a concentration of 0.01-5% w/w based on the total weight of the aqueous solution.

Preferably, the filtration phase iii) is carried out at a pressure of 1-8 bar, more preferably 1.5-6 bar, even more preferably about 5 bar.

Preferably, the process of the invention is implemented in a closed circuit plant in which a filtration system comprising the filter is connected to mixing tanks with thermostat jacket upstream and to a filling machine downstream, said tanks being adapted to execute the pre-treatment of the aqueous solution. Therefore, the present invention further relates to such a plant.

In preferred aspects, the process for the preparation of sterile products further comprises the phase of:
iv) packaging the filtered product in vials, filling such vials with the filtered product to the desired volume, capping the vials with a cap, preferably made of butyl elastomer and with aluminum ring.

In preferred embodiments, the products obtainable by the process according to the present invention are cosmetic products or medical devices, in particular to be used as adjuvants to reinforce and promote hair growth, to revitalize atonic and dull skin, to counteract the formation of expression wrinkles and deep wrinkles, to improve the appearance of skin spots, to biostimulate the regrowth of connective tissue, for the volume increase of soft tissues or to aid the action of lipolytic treatments.

In another aspect, the present invention also relates to the cosmetic or medical use of the sterile products obtainable by the process described above, as adjuvants in cosmetic mesotherapy or in medical treatments.

Said sterile products obtainable according to the invention preferably comprise hydrosoluble bioactive raw materials, such as hyaluronic acid, amino acids, vitamins, oligopeptides, sh-polypeptides, chelating agents, and further hydrosoluble components such as basic and/or acidic acidity regulators. Said products preferably further comprise preservatives and bactericides, and optionally components soluble in glycol or glycerol, such as terpenes and essential oils.

Sterile products having the following compositions are particularly preferred:
(CI=hydrosoluble component; C=preservative; CG=glycerin-soluble component).
   I. sterile mesotherapy bio-revitalizing product, suitable for scalp treatment, to strengthen and promote hair growth through the improvement of microcirculation and the stimulation of endogenous processes:

| | % w/w | CLASS |
|---|---|---|
| WFI | As needed | |
| | to 100 | |
| Hyaluronic Acid 500Da-3MDa | 1-3 | CI |
| Phenoxyethanol, ethylhexylglycerin | 0.01-5 | C |
| Sodium Hydroxide | 0.01-5 | CI |
| Succinic Acid | 0.01-5 | CI |
| Ferrous Gluconate | 0.01-5 | CI |
| Water, Hydrolyzed Keratin | 0.01-5 | CI |
| Panthenol | 0.01-5 | CI |
| Pyridoxine hydrochloride | 0.01-5 | CI |
| Thiamin | 0.01-5 | CI |
| Biotin | 0.01-5 | CI |
| Sh-Polypeptide-11 | 0.01-5 | CI |
| Sh-Polypeptide-1 | 0.01-5 | CI |
| Sh-Oligopeptide-2 | 0.01-5 | CI |
| Sh-Polypeptide-3 | 0.01-5 | CI |
| Sh-Polypeptide-9 | 0.01-5 | CI |
| Copper Tripeptide-1 | 0.01-5 | CI |
| Sh-Oligopeptide-4 | 0.01-5 | CI |
| Arginine | 0.01-5 | CI |
| Citric Acid | 0.01-5 | CI |
| Water, pentylene glycol polysaccharide | 0.01-5 | CI |
| cladosiphon novaecaledoniae, phenoxyethanol | | |
| Swertia japonicaextract, butylene glycol | 0.01-5 | CI |

II. sterile mesotherapy product, which can be used as an adjuvant biorevitalizing agent in the treatment of atonic skin and expression wrinkles, through the improvement of microcirculation and the stimulation of endogenous processes:

| | % w/w | CLASS |
|---|---|---|
| WFI | As needed | CI |
| | to 100 | |
| Hyaluronic Acid 500Da-3MDa | 6-10 | CI |
| Phenoxyethanol, ethylhexylglycerin | 0.01-5 | C |
| Sodium Hydroxide | 0.01-5 | CI |
| Succinic Acid | 0.01-5 | CI |
| Proline | 0.01-5 | CI |
| Lysine | 0.01-5 | CI |
| Leucine | 0.01-5 | CI |
| Isoleucine | 0.01-5 | CI |
| Hydroxyproline | 0.01-5 | CI |
| Glycine | 0.01-5 | CI |
| Valine | 0.01-5 | CI |
| Threonine | 0.01-5 | CI |
| Phenylalanine | 0.01-5 | CI |
| Methionine | 0.01-5 | CI |
| Glutamic Acid | 0.01-5 | CI |
| Arginine | 0.01-5 | CI |

III. sterile mesotherapy biorevitalizing product, which can be used as an adjuvant in the prevention and treatment of expression lines and deep wrinkles, through the improvement of microcirculation and the stimulation of endogenous processes:

| | % w/w | CLASS |
|---|---|---|
| WFI | As needed | CI |
| | to 100 | |
| Hyaluronic Acid 500Da-3MDa | 4-8 | CI |
| Phenoxyethanol, ethylhexylglycerin | 0.01-5 | C |
| Sodium Chloride | 0.01-5 | CI |
| Sodium Hydroxide | 0.001-0.5 | CI |
| Acetyl Tetrapeptide-11 | 0.001-0.5 | CI |
| Pentapeptide-3 | 0.001-0.5 | CI |
| Oligopeptide-34 | 0.001-0.5 | CI |
| Copper Tripeptide-34 | 0.001-0.5 | CI |
| Tripeptide-29 | 0.001-0.5 | CI |
| Myristoyl Pentapeptide-8 | 0.001-0.5 | CI |
| Pal-Tetrapeptide-7 | 0.001-0.5 | CI |

IV. sterile mesotherapy biorevitalizing product, which can be used as an adjuvant in the treatment of skin spots of different etiology, through the improvement of microcirculation and the stimulation of endogenous processes:

| | % w/w | CLASS |
|---|---|---|
| WFI | As needed | CI |
| | to 100 | |
| Hyaluronic Acid 500Da-3MDa | 2-5 | CI |
| Phenoxyethanol, ethylhexylglycerin | 0.01-5 | C |
| Sodium Hydroxide | 0.01-5 | CI |
| Rutin | 0.001-0.5 | CI |
| Ellagic acid | 0.01-5 | CI |
| Procysteine | 0.01-5 | CI |
| Betula pendula Roth et/aut B. pubescens Ehrh. | 0.01-5 | CI |
| Lecithin, Camellia sinensisleaf extract | 0.01-5 | CI |
| Salvia officinalisleaf extract | 0.01-5 | CI |
| Melissa officinalisleaf extract, maltodextrin, water | 0.01-5 | CI |
| Rosmarinus officinalisextract | 0.001-0.5 | CI |
| Water, glycerin, Caprylyl Glycol and Acetyl | 0.01-5 | CI |
| Tetrapeptide-2 | | |
| glycerin, Water, Vitis viniferaleaf extract | 0.01-5 | CI |
| Tranexamic acid | 0.01-5 | CI |
| Salicylic Acid | 0.01-5 | CI |
| Tetrahydropyranyloxy phenol | 0.01-5 | CI |
| Sodium DNA | 0.01-5 | CI |
| Potassium Azeloyl Diglycinate | 0.01-5 | CI |
| Oligopeptide-34 | 0.001-0.5 | CI |
| Aminoethylphosphinic acid, butylene glycol and | 0.01-5 | CI |
| water | | |
| Glutathione | 0.01-5 | CI |
| Sodium phytate | 0.01-5 | CI |

V. sterile mesotherapy biorevitalizing product, adapted to assist the treatment of dull and atonic skin, through the improvement of microcirculation and the stimulation of endogenous processes:

| | % w/w | CLASS |
|---|---|---|
| WFI | As needed | CI |
| | to 100 | |
| Hyaluronic Acid 500 Da-3 MDa | 2-6 | CI |
| Phenoxyethanol, ethylhexylglycerin | 0.01-5 | C |
| Sodium Hydroxide | 0.001-0.5 | CI |
| Ascorbyl glucoside | 0.01-5 | CI |
| Curcumin | 0.001-0.5 | CG |
| Riboflavin | 0.001-0.5 | CI |
| Ferulic acid | 0.01-5 | CI |
| Eugenol | 0.01-5 | CG |
| Safranal | 0.01-5 | CG |
| Glycerin | 0.01-5 | CG |
| Propylene glycol | 0.01-5 | CI |

VI. sterile mesotherapy biorevitalizing product, used as an adjuvant in the prevention and treatment of localized adiposities, through the improvement of microcirculation and the stimulation of endogenous processes:

| | % w/w | CLASS |
|---|---|---|
| WFI | As needed | CI |
| | to 100 | |
| Hyaluronic Acid 500 Da-3 MDa | 0.1-3 | CI |
| Phenoxyethanol, ethylhexylglycerin | 0.01-5 | C |
| Sodium Chloride | 0.01-5 | CI |
| Sodium Deoxycholate | 0.01-5 | CI |
| Citric Acid | 0.001-0.5 | CI |

VII. sterile product comprising 0.5% w/w hyaluronic acid having molecular weight 1.6 MDa:

| | % w/w | CLASS |
|---|---|---|
| WFI | As needed | CI |
| | to 100 | |
| Hyaluronic Acid 500 Da-3 MDa | 0.1-1 | CI |
| Phenoxyethanol, ethylhexylglycerin | 0.01-5 | C |
| Sodium Hydroxide | 0.001-0.5 | CI |

where “% w/w” stands for “% by weight, based on the weight of the product,” and “WFI” stands for Water for Injection.

Preferably, phase i. of the present process of providing the aqueous solution, comprises the following sub-phases:
i.a) mixing in water the hydrosoluble compounds of the aqueous solution, obtaining a first solution (A);
i.b) optionally mixing any preservatives and/or compounds which are soluble in glycol or glycerin, obtaining a second solution (B) and adding the second solution B to the first solution A, obtaining a third solution (C);
i.e) adding the thermolabile and/or bioactive raw material to the third solution C if present, or to the first solution A, obtaining the aqueous solution comprising the thermolabile and/or bioactive raw material of phase i);
in which the sub-phases i.a)-i.e) are carried out at room temperature.

It is to be understood that all possible combinations of the preferred aspects of the phases and sub-phases of the process, as well as of the ingredients of the sterile products, as indicated above, are also described and therefore similarly preferred.

It is further to be understood that all the aspects identified as preferred and advantageous for the process are likewise to be considered preferred and advantageous also for the sterile products and the uses thereof.

The following examples of embodiments of the present invention are given below by way of non-limiting illustration.

### EXAMPLES

### Example 1

A solution comprising hyaluronic acid (molecular weight: 1.6 MDa) at 0.5% w/w and NaCl 0.8% w/w was subjected to several heat treatments to monitor the effect of heat on the weight loss of the solution, due to water evaporation.

The six samples were subjected to increasing temperatures, under stirring, for 4 hours. Viscosity (Brookfield DV2T viscometer) and percentage weight loss were measured at room temperature (20° C.) or at the treatment temperature.

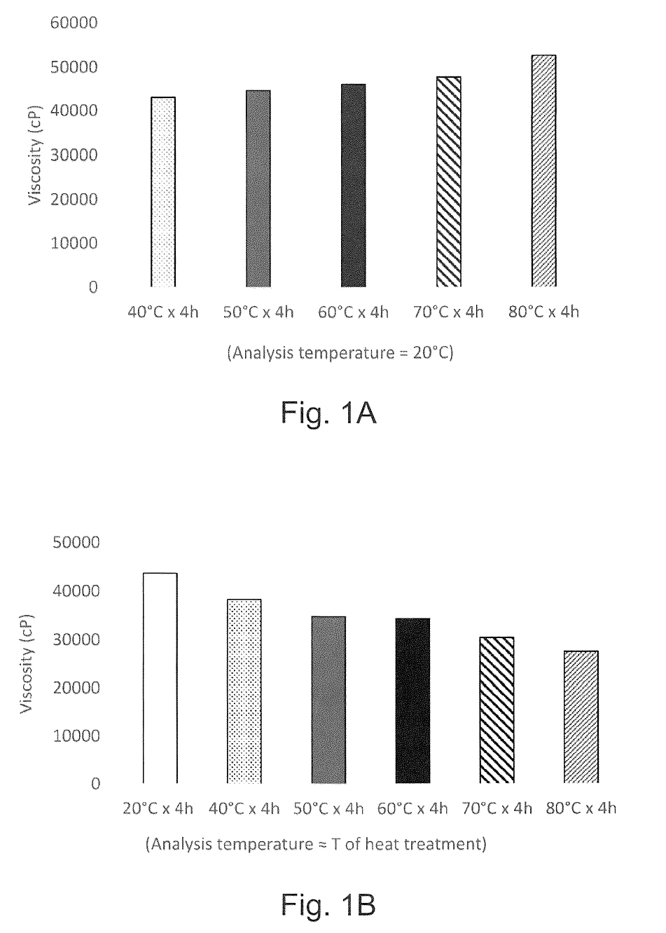

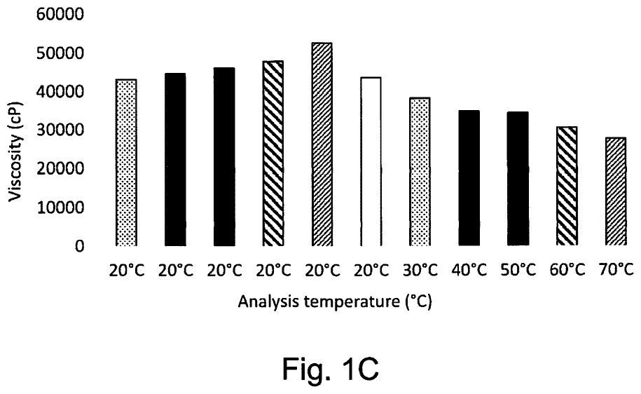

The results are reported in Tables 1a (viscosity measured at room temperature) and 1b (viscosity measured at the temperature approximately equal to the treatment temperature). FIGS. 1A and 1B graphically show the results reported in tables 1a and 1b, respectively.

TABLE 1a

| | | | | | | | | Percent |
|---|---|---|---|---|---|---|---|---|
| | | | | | | | Post- | age % |
| | Pre- | Analysis | | Viscosity | | Starting | treatment | weight |
| | treatment | T (° C.) | Spindle | (cP) | | weight | weight | loss |
| 1) | 40° C. × 4 h | 20° C. | 06(6) | 43150 ± 500 | cP | 100 | 100 | 0 |
| 2) | 50° C. × 4 h | 20° C. | 06(6) | 44700 ± 500 | cP | 100 | 99.61 | 0.39% |
| 3) | 60° C. × 4 h | 20° C. | 06(6) | 46150 ± 500 | cP | 100 | 96.53 | 3.47% |
| 4) | 70° C. × 4 h | 20° C. | 07(7) | 47800 ± 2000 | cP | 100 | 95.02 | 4.98% |
| 5) | 80° C. × 4 h | 20° C. | 07(7) | 52600 ± 2000 | cP | 100 | 90.69 | 9.31% |

TABLE 1b

| | | | | | Reduction % |
|---|---|---|---|---|---|
| | | | | | in viscosity |
| | Pre- | Analysis | | Viscosity | with respect |
| | treatment | T (° C.) | Spindle | (cP) | to (1) |
| (1) | 20° C. × 4 h | 20° C. | 06(6) | 43700 ± 500 cP | — |
| (2) | 40° C. × 4 h | 30° C. | 06(6) | 38300 ± 500 cP | 12.35% |
| (3) | 50° C. × 4 h | 40° C. | 06(6) | 34850 ± 500 cP | 20.25% |
| (4) | 60° C. × 4 h | 50° C. | 06(6) | 34400 ± 500 cP | 21.28% |
| (5) | 70° C. × 4 h | 60° C. | 06(6) | 30550 ± 500 cP | 30.09% |
| (6) | 80° C. × 4 h | 70° C. | 06(6) | 27600 ± 500 cP | 36.84% |

The reduction in viscosity of table 1b was calculated with respect to the viscosity of the sample (1) kept under stirring at room temperature.

It is known that as the temperature increases, the viscosity of a solution containing hyaluronic acid decreases. For example, it is known that subjecting a solution containing hyaluronic acid to temperatures up to 70° C. does not result in the degradation of the linear and transverse polymer chains, rather it results in a decrease in the viscosity such as to allow filtration through 0.22 μm pores.

However, such a decrease in viscosity is temporary, as at room temperature the solution returns to the same viscosity value as prior to heating, if not higher if the solvent (water) has evaporated.

In fact, Table 1a and FIG. 1a show a higher viscosity (measured at room temperature of 20° C.) after treatment at higher temperatures, due to greater solvent evaporation.

Conversely, Table 1b and FIG. 1b show a lower viscosity at higher temperatures (measured at temperatures close to the treatment temperature).

FIG. 1C collects the results in FIGS. 1A and 1B together to provide an overview of the results.

The pre-treatment at a temperature of 60° C. is particularly advantageous in that at such a temperature the viscosity is reduced to a satisfactory level, with weight loss <5%. Therefore, the viscosity of the products kept at 60° C. is preferable to facilitate the filtration of the solution through 0.22 μm pores; moreover at 60° C. there is no evaporation of water, which would lead to an increase in the concentration of the polymer in solution and no degradation of the hyaluronic acid chains. Therefore, there is a particularly high advantage in terms of filterability and shelf life and stability of the product by subjecting the solution to a pre-treatment and to filtration at a temperature of about 60° C.

### Example 2

The effect of the same heat and stirring pre-treatment (phase ii), according to a preferred aspect of the process of the invention, on the viscosity of different products comprising an increasing content of hyaluronic acid (HA) and, therefore, increasing viscosity was tested. The solutions tested are: Sample A with 1% w/w HA 0.5 MDa and 0.1% w/w HA 1 MDa; Sample C with 1.5% w/w HA 0.5 MDa and 0.1% w/w HA 1 MDa; Sample D with 1.75% w/w HA 0.5 MDa and 0.1% w/w HA 1 MDa; Sample E with 2% w/w HA 0.5 MDa and 0.1% w/w HA 1 MDa; Sample G with 2.5% w/w HA 0.5 MDa and 0.1% w/w HA 1 MDa.

The samples were stirred overnight at room temperature, then allowed to rest overnight at room temperature and finally heated to about 60° C. with stirring for 2 hours.

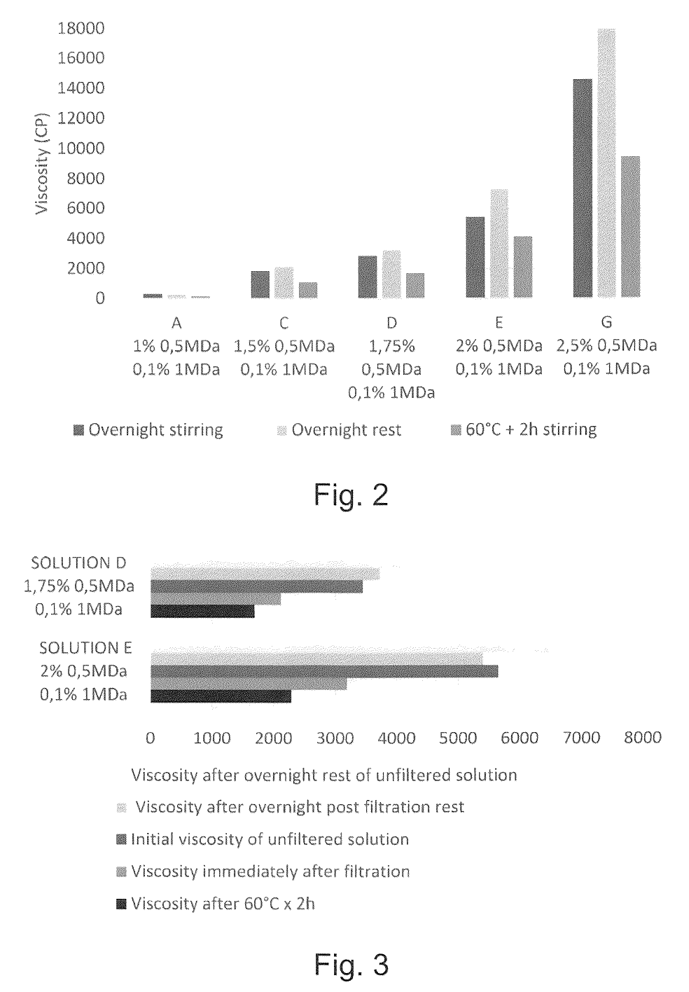

The viscosities of the different samples were measured after each treatment, at the same temperature as the treatment. The measurement results (viscosity n and sample temperature at viscosity measurement) are shown in table 2 below and FIG. 2.

TABLE 2

| | Day 2 | | |
|---|---|---|---|
| Day 1 | Overnight | Day 3 | 60° C. + 2 h |
| Sample (200 g) | stirring | Overnight rest | stirring |
| A | η = 280 cP | η = 320 cP | η = 160 cP |
| 1% HA 0.5 MDa | t = 21.1° C. | t = 21.1° C. | t = 60.5° C. |
| 0.1% HA 1 MDa | | | |
| C | η = 1840 cP | η = 2150 cP | η = 1110 cP |
| 1.5% HA | t = 21.9° C. | t = 21.9° C. | t = 61.6° C. |
| 0.5 MDa | | | |
| 0.1% HA 1 MDa | | | |
| D | η = 2860 cP | η = 3310 cP | η = 1710 cP |
| 1.75% HA | t = 21.9° C. | t = 21.9° C. | t = 61.9° C. |
| 0.5 MDa | | | |
| 0.1% HA 1 MDa | | | |
| E | η = 5410 cP | η = 7330 cP | η = 4110 cP |
| 2% HA 0.5 MDa | t = 20.9° C. | t = 20.9° C. | t = 60.9° C. |
| 0.1% HA 1 MDa | | | |
| G | η = 14560 cP | η = 17980 cP | η = 9440 cP |
| 2.5% HA | t = 20.7° C. | t = 20.7° C. | t = 61.1° C. |
| 0.5 MDa | | | |
| 0.1% HA 1 MDa | | | |

The percentage viscosity change measured in the three stages of analysis (room temperature and overnight stirring, room temperature after overnight rest, 60° C. after 2 hours stirring) is consistent in the different samples: after one night (overnight) at rest, there is an increase in viscosity of the solutions from 14% to 35% with respect to the sample left for one night under stirring; after heat treatment for two hours, there is a reduction in viscosity from 50% to 56% with respect to the sample left one night at rest.

An exponential, reproducible and predictable correlation is apparent for the three stages of analysis between the samples with increasing hyaluronic acid content.

### Example 3

The filterability of a cosmetic grade hyaluronic acid with a molecular weight of 1.6 MDa was tested through a 0.22 μm classic filtering cartridge at room temperature. A clogging of the filter was observed on the external surface, as if a gel patina had formed which did not allow the solution to pass through the pleats of the cartridge. Furthermore, the viscosity of the low amount of filtered product was much lower than that of the initial sample. This indicates that, without treatment of the pre-filtration sample and without the use of suitable filters, the solution is not filterable and therefore not sterilizable by filtration.

### Example 4

The filterability of solutions D and E of Example 2, comprising HAs of molecular weight and concentration similar to sterile products employable in mesotherapy, was tested with a filter (1) comprising a support membrane (12a, 12b) on each side of a polyethersulfone filtering membrane (11) (see the filter in FIG. 7), according to preferred aspects of the present invention.

The viscosities of the solutions analyzed in the various phases of the test were measured:
initial viscosity of the unfiltered solution
viscosity after 2 hours under stirring at about 60° C.
viscosity immediately after filtration
post-filtration viscosity, after 1 night (overnight) at rest
viscosity of the unfiltered solution after 1 night (overnight) at rest
post-filtration viscosity, after 2 nights (overnight) at rest
viscosity of the unfiltered solution after 2 nights (overnight) at rest

The viscosity values measured for the filtered samples and for unfiltered comparison samples in each phase are shown in table 3 below (the temperature of the samples at the viscosity measurement is shown in square brackets).

In the unfiltered solutions (comparison solutions) an increase in viscosity is observed over time. Surprisingly, the pre-treated and filtered solutions according to the process of the present invention, at the same pre-treatment temperature and with the multilayer pleated filter, do not exhibit any significant change in viscosity over time at room temperature, and maintain a viscosity substantially identical to the pre-filtration sample at room temperature in the days following the test (see FIG. 3 “Initial viscosity of unfiltered solution” vs. “Viscosity after overnight post-filtration rest”).

TABLE 3

| | Initial | | | | | |
|---|---|---|---|---|---|---|
| | viscosity | Viscosity | | Viscosity | Viscosity | Viscosity |
| | of the | after 2 h | | immediately | after | after |
| | unfiltered | stirring at | | after | overnight | overnight |
| | solution | 60° C. | | filtration | rest | rest |
| Product | (day 0) | (day 0) | Filter_ing | (day 0) | (day +1) | (day +2) |
| Solution E | 5650 cP | 2290 cP | yes | 3190cP | 5000 cP | 5400 cP |
| | [22° C.] | [62° C.] | | [32.6° C.] | [21.3° C.] | [19.5° C.] |
| Solution D | 3450 cP | 1690 cP | yes | 2120cP | 3560 cP | 3730 cP |
| | [22° C.] | [59° C.] | | [32° C.] | [21.6° C.] | [19.4° C.] |
| Comparison | 5650 cP | 2290 cP | no | — | 6360 cP | 7000 cP |
| Solution E | [22° C.] | [62° C.] | | | [21.5° C.] | [19° C.] |
| (not filtered) | | | | | | |
| Comparison | 3450 cP | 1690 cP | no | — | 4560 cP | 4530 cP |
| solution D | [22° C.] | [59° C.] | | | [19.6° C.] | [19.0° C.] |
| (not filtered) | | | | | | |

The heated and unfiltered solution provides a higher viscosity value at room temperature as compared to the initial one, with a tendency to increase over time (see the viscosity at day +1 and at day +2 of the unfiltered comparison solutions), presumably for undesired chain-chain interactions stimulated by the heat treatment; on the other hand, the filtered solution provides a viscosity value at room temperature which is very similar to the initial unfiltered one and such a value tends to remain constant over time, highlighting a greater stability of the product.

In FIG. 3, the viscosities are compared in the various phases of the test, pre- and post-filtration.

In particular, it can be seen that the initial viscosity and the viscosity after filtration are almost the same. The viscosity of the unfiltered solution is instead higher after 2 days as compared to the initial one, while the viscosity of the filtered solution after 2 days is more stable.

After filtration, not only the rheological features of the initial solution are maintained, but a more stable compound is obtained over time as compared to the unfiltered one.

### Example 5

Further tests were carried out with multilayer pleated cartridge filters (2), according to preferred aspects of the present invention (see filter (2) in FIGS. 8 and 9), which have a structure similar to the filter (1) used in example 4, but with even more reinforced and thus spaced pleats (20a, 20b), by virtue of the presence of two stiff lattices (23a and b, 23′a and b) on each side of the filtering membrane (21), after the support membranes (22a and b), which reduce the clogging of the cartridge. In fact, the filtration rate through these filters was even higher than the filtration rate through the filter (1) of example 4. The filtered solution was sterile. The filters employed in the present example are particularly suitable for the filtration of solutions with high viscosity.

### Example 6

Filtration tests were carried out at different operating pressures. With an operating pressure of 1.5 bar, there is a loss of filtered product of 64% on 800 g. At 5 bar, however, advantages are obtained in terms of both greater filtrate flow and waste minimization, which amounts to 2% on 800 g.

### Example 7

The effects of sterilization by filtration, according to the present invention, were compared to the effects of sterilization in autoclave, for the following formulations, where “% w/w” stands for “% by weight, on the weight of the product and “WFI” stands for Water for Injection.
MESO HAIR REV01: sterile mesotherapy bio-revitalizing product, suitable for scalp treatment to strengthen and promote hair growth through the improvement of microcirculation and the stimulation of endogenous processes. The qualitative-quantitative composition of the product is shown in table 4.

TABLE 4

| MESO HAIR REV01 | % w/w |
|---|---|
| WFI | As |
| | needed |
| | to 100 |
| Hyaluronic Acid 500 Da-3 MDa | 2 |
| Phenoxyethanol, ethylhexylglycerin | 1 |
| Sodium Hydroxide | 0.01 |
| Succinic Acid | 1 |
| Ferrous Gluconate | 0.01 |
| Water, Hydrolyzed Keratin | 0.01 |
| Panthenol | 0.01 |
| Pyridoxine hydrochloride | 0.01 |
| Thiamin | 0.01 |
| Biotin | 0.01 |
| Sh-Polypeptide-11 | 0.01 |
| Sh-Polypeptide-1 | 0.01 |
| Sh-Oligopeptide-2 | 0.01 |
| Sh-Polypeptide-3 | 0.01 |
| Sh-Polypeptide-9 | 0.01 |
| Copper Tripeptide-1 | 0.01 |
| Sh-Oligopeptide-4 | 0.01 |
| Arginine | 0.01 |
| Citric Acid | 0.01 |
| Water, pentylene glycol | 0.01 |
| polysaccharide cladosiphon novaecaledoniae, | |
| phenoxyethanol | |
| Swertia japonica extract, butylene glycol | 0.01 |

MESO SUCCINIC+HA+AA REV00: sterile mesotherapy product, used as a generic biorevitalizing agent in the treatment of atonic skin and expression wrinkles, through the improvement of microcirculation and the stimulation of endogenous processes. The qualitative-quantitative composition of the product is shown in table 5.

TABLE 5

| MESO SUCCINIC + HA + AA REV00 | % w/w |
|---|---|
| WFI | As |
| | needed |
| | to 100 |
| Hyaluronic Acid 500 Da-3 MDa | 8 |
| Phenoxyethanol, ethylhexylglycerin | 1 |
| Sodium Hydroxide | 0.01 |
| Succinic Acid | 1 |
| Proline | 0.01 |
| Lysine | 0.01 |
| Leucine | 0.01 |
| Isoleucine | 0.01 |
| Hydroxyproline | 0.01 |
| Glycine | 0.01 |
| Valine | 0.01 |
| Threonine | 0.01 |
| Phenylalanine | 0.01 |
| Methionine | 0.01 |
| Glutamic Acid | 0.01 |
| Arginine | 0.01 |

MESO ANTIAGE-C REV02: sterile mesotherapy biorevitalizing product, used as an adjuvant in the prevention and treatment of expression lines and deep wrinkles, through the improvement of microcirculation and the stimulation of endogenous processes. The qualitative-quantitative composition of the product is shown in table 6.

TABLE 6

| MESO ANTIAGE-C REV02 | % w/w |
|---|---|
| WFI | As |
| | needed |
| | to 100 |
| Hyaluronic Acid 500 Da-3 MDa | 6 |
| Phenoxyethanol, ethylhexylglycerin | 1 |
| Sodium Chloride | 0.5 |
| Sodium Hydroxide | 0.001 |
| Acetyl Tetrapeptide-11 | 0.001 |
| Pentapeptide-3 | 0.001 |
| Oligopeptide-34 | 0.001 |
| Copper Tripeptide-34 | 0.001 |
| Tripeptide-29 | 0.001 |
| Myristoyl Pentapeptide-8 | 0.001 |
| Pal-Tetrapeptide-7 | 0.001 |

MESO WHITE REV01: cosmetic mesotherapy biorevitalizing product, used as an adjuvant in the treatment of skin spots of different etiology, through the improvement of microcirculation and the stimulation of endogenous processes. The qualitative-quantitative composition of the product is shown in table 7.

TABLE 7

| MESO WHITE REV01 | % w/w |
|---|---|
| WFI | As |
| | needed |
| | to 100 |
| Hyaluronic Acid 500 Da-3 MDa | 3 |
| Phenoxyethanol, ethylhexylglycerin | 1 |
| Sodium Hydroxide | 0.01 |
| Rutin | 0.001 |
| Ellagic acid | 0.01 |
| Procysteine | 0.01 |
| Betula pendula Roth et/aut B. pubescens Ehrh. | 0.01 |
| Lecithin (syn. phosphatidylcholine), Camellia sinensis | 0.01 |
| leaf extract | |
| Salvia officinalis leaf extract | 0.01 |
| Melissa officinalis leaf extract, maltodextrin, water | 0.01 |
| Rosmarinus officinalis extract | 0.001 |
| Water, glycerin, Caprylyl Glycol and Acetyl Tetrapeptide- | 0.01 |
| 2 | |
| glycerin, Water, Vitis vinifera leaf extract | 0.01 |
| Tranexamic acid | 0.01 |
| Salicylic Acid | 0.01 |
| Tetrahydropyranyloxy phenol | 0.01 |
| Sodium DNA | 0.01 |
| Potassium Azeloyl Diglycinate | 0.01 |
| Oligopeptide-34 | 0.001 |
| Aminoethylphosphinic acid, butylene glycol and water | 0.01 |
| Glutathione | 0.01 |
| Sodium phytate | 0.01 |

MESO ANTIOX+HA REV01: sterile mesotherapy biorevitalizing product, intended to assist in the treatment of dull and atonic skin, through the improvement of microcirculation and the stimulation of endogenous processes. The qualitative-quantitative composition of the product is shown in table 8.

TABLE 8

| MESO ANTIOX + HA REV01 | % w/w |
|---|---|
| WFI | As needed |
| | to 100 |
| Hyaluronic Acid 500 Da-3 MDa | 4 |
| Phenoxyethanol, ethylhexylglycerin | 1 |
| Sodium Hydroxide | 0.001 |
| Ascorbyl glucoside | 0.01 |
| Curcumin | 0.001 |
| Riboflavin | 0.001 |
| Ferulic acid | 0.01 |
| Eugenol | 0.01 |
| Safranal | 0.01 |
| Glycerin | 5 |
| Propylene glycol | 0.01 |

SODIUM DEOXYCHOLATE REV02: sterile mesotherapy bio-revitalizing product, used as an adjuvant in the prevention and treatment of localized adiposities, through the improvement of microcirculation and the stimulation of endogenous processes. The qualitative-quantitative composition of the product is shown in table 9.

TABLE 9

| SODIUM DEOXYCHOLATE REV02 | % w/w |
|---|---|
| WFI | As needed |
| | to 100 |
| Hyaluronic Acid 500 Da-3 MDa | 1 |
| Phenoxyethanol, ethylhexylglycerin | 1 |
| Sodium Chloride | 0.5 |
| Sodium Deoxycholate | 5 |
| Citric Acid | 0.001 |

HA 1.6 MDa 0.5% cosmetic grade formulation comprising 0.5% w/w hyaluronic acid having molecular weight 1.6 MDa. The qualitative-quantitative composition of the product is shown in table 10.

TABLE 10

| HA 1.6 MDa 0.5% | % w/w |
|---|---|
| WFI | As needed |
| | to 100 |
| Hyaluronic Acid 1.6 MDa | 0.5 |
| Phenoxyethanol, ethylhexylglycerin | 1 |
| Sodium Hydroxide | 0.001 |

In order to evaluate the advantages of sterilization by filtration according to the invention as compared to that in an autoclave, the following effects are taken into account, which are related to variations in the qualitative-quantitative composition:
Color change, due to the degradation of thermolabile substances. Other thermolabile substances such as preservatives do not produce visible consequences, but lose the effectiveness and original structure thereof
Variation of pH outside the tolerance range (for mesotherapy formulations, except those for around the eye or other specific applications, it is 7.30±1) to be attributed to the release of nitrogenous compounds, the oxidation of alcohols or cleavage of intra-molecular bonds;
Decrease in viscosity, due to the degradation of hyaluronic acid polymer chains which, despite being at the same initial concentration, lose their viscosifying power and their resistance to hyaluronidases (the viscosity acceptability range is provided by the instrument itself);
Unknown and unwanted interactions between products and by-products;
Release of irritants, such as nitrogenous compounds;
Degradation of thermolabile raw materials, which lose their effectiveness (i.e., preservatives, peptides, vitamins . . . );
Formation of floccules or precipitates, due to variation in pH and/or degradation of the raw material.

In particular, the effects of sterilization in autoclave, with respect to sterilization by filtration according to the present invention, are shown here for each formulation on the following parameters:
Pre-sterilization pH vs. post-sterilization pH
Solution color
Pre-sterilization viscosity vs. post-sterilization viscosity
Presence of precipitate

After sterilization by filtration according to the present invention, the solutions do not undergo significant chemical-physical variations with respect to the starting solutions.

The variations in pH are shown in table 11 and the variations in change in table 12, the latter measured in the HA 1.6 MDa 0.5% formulation, representative of the variation in viscosity of all the other formulations.

TABLE 11

| | Pre- | Sterilization | Sterilization |
|---|---|---|---|
| | sterilization | in autoclave | by filtration |
| MESO HAIR | pH 7.31 | pH 7.47 | pH 7.56 |
| REV01 | | | |
| (composition in | | | |
| Table 4) | | | |
| MESO SUCCINIC + | pH 7.66 | pH 7.97 | pH 7.98 |
| HA + AA REV00 | | | |
| (composition in | | | |
| Table 5) | | | |
| MESO ANTIAGE- | pH 7.30 | pH 8.65 | pH 8.11 |
| C REV02 | | | |
| (composition in | | | |
| Table 6) | | | |
| HA 1.6 MDa 0.5% | pH 7.13 | pH 7.38 | pH 6.86 |
| (composition in | | | |
| Table 10) | | | |
| MESO WHITE | pH 7.14 | pH 6.32 | pH 6.92 |
| REV01 | | | |
| (composition in | | | |
| Table 7) | | | |
| MESO ANTIOX + | pH 7.13 | pH 7.36 | pH 7.44 |
| HA REV01 | | | |
| (composition in | | | |
| Table 8) | | | |
| SODIUM | pH 7.84 | pH 7.88 | pH 7.80 |
| DEOXYCHOLATE | | | |
| REV02 | | | |
| (composition in | | | |
| Table 9) | | | |

TABLE 12

| | Pre- | Sterilization | Sterilization |
|---|---|---|---|
| | sterilization | in autoclave | by filtration |
| HA 1.6 MDa 0.5% | 1760 cP | 700 cP | 1605 cP |
| | [22.1° C.] | [24.1° C.] | [24° C.] |

MESO HAIR REV01 contains thermolabile substances, whereby after exposure to heat in the autoclave a change in color is noted, as well as fragmentation of the hyaluronic acid chains, ineffectiveness of the preservative system and other ingredients and likely release of by-products and/or irritants.

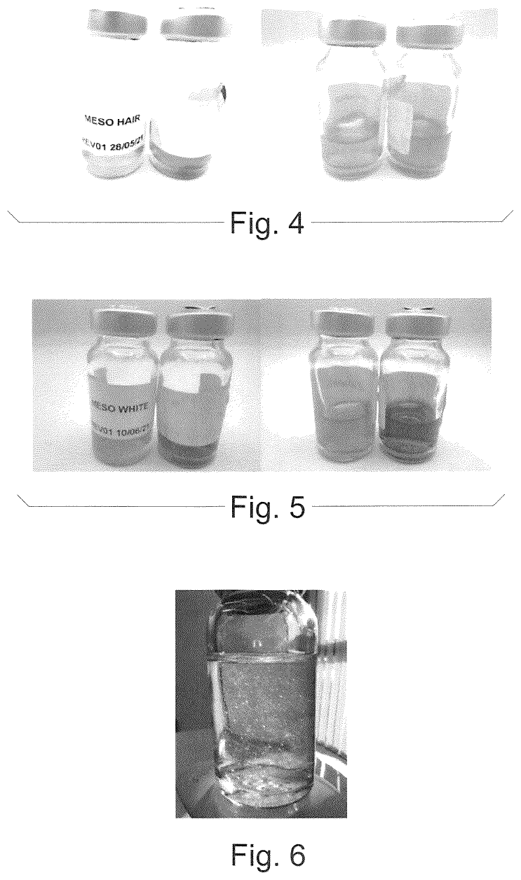

In FIG. 4, the colorimetric comparison between a sample of MESO HAIR REV01 sterilized by filtration according to the method of the present invention (left) and a sample of the same formulation sterilized in autoclave (right) is shown.

MESO SUCCINIC+HA+AA REV00 contains thermolabile substances, whereby after exposure to heat in the autoclave fragmentation of the hyaluronic acid chains is noted, as well as ineffectiveness of the preservative system and other ingredients and probable release of by-products and/or irritants.

MESO ANTIAGE-C REV02 contains thermolabile substances, whereby after exposure to heat in an autoclave, an increase in pH is noted, as well as fragmentation of the hyaluronic acid chains, ineffectiveness of the preservative system and other ingredients and likely release of by-products and/or irritants.

MESO WHITE REV01 contains thermolabile substances, whereby after exposure to heat in the autoclave a decrease in pH is noted, as well as color variation, fragmentation of the hyaluronic acid chains, ineffectiveness of the preservative system and other ingredients and likely release of by-products and/or irritants.

In FIG. 5, the colorimetric comparison between a sample of MESO WHITE REV01 sterilized by filtration according to the method of the present invention (left) and a sample of the same formulation sterilized in autoclave (right) is shown.

MESO ANTIOX+HA REV01 contains thermolabile substances, whereby after exposure to heat in the autoclave a change in color is noted, as well as fragmentation of the hyaluronic acid chains, ineffectiveness of the preservative system and other ingredients and likely release of by-products and/or irritants.

SODIUM DEOXYCHOLATE REV02 contains thermolabile substances, whereby after exposure to heat in the autoclave, formation of white precipitate is noted, as well as fragmentation of the hyaluronic acid chains, ineffectiveness of the preservative system and other ingredients and likely release of by-products and/or irritants (see FIG. 6).

Table 13 below summarizes the effects found after sterilization in autoclave of the different formulations.

TABLE 13

| | | | | Release |
|---|---|---|---|---|
| | Color | Increase/ | Decreased | of by- |
| | change | decrease | viscosity | products |
| | after | pH after | after | and/or |
| Formulation | autoclave | autoclave | autoclave | irritants |
| MESO HAIR - | yes | no | yes | yes |
| REV01 | | | | |
| MESO SUCCINIC + | no | no | yes | yes |
| HA + AA - | | | | |
| REV00 | | | | |
| MESO ANTIAGE- | no | yes | yes | yes |
| C - REV02 | | | | |
| MESO WHITE - | yes | yes | yes | yes |
| REV01 | | | | |
| MESO ANTIOX + | yes | no | yes | yes |
| HA - REV01 | | | | |
| SODIUM | Yes | no | yes | yes |
| DEOXYCHOLATE | (precipitated) | | | |
| REV02 | | | | |

None of the undesirable effects reported after sterilization in autoclave was detected following sterilization by filtration according to the present invention.

The process of the present invention thus provides sterile products of high quality and safety, and ensures that the qualitative-quantitative composition of the product after filtration is identical to the initial one.

### Example 8

For the production scale-up, 2 kg of MESO ANTIAGE-C REV02 (see the composition in table 6), which, having the greatest viscosity, represents the most critical product among the formulations considered, were filtered through a 5″ PES 66 filter; the product was completely filtered with continuous flow in about 2′. It is therefore estimated that with the same 30″ filter (1.8 m²), 20-30 kg of a similar product can be filtered without clogging.

### Example 9

Sterile products comprising but not limited to the formulations analyzed in the preceding examples, are obtained by the following preparation process:
1. mixing the hydrosoluble components in water, at about 20° C. (solution A)
2. adding a premixed solution B, comprising preservatives and raw materials soluble in glycol and/or glycerol, to solution A (solution C)
3. adding hyaluronic acid to solution C, at about 20° C. (solution D)
4. measuring the pH of solution D
5. stirring overnight at 100-5,000 rpm at 20° C.
6. measuring the viscosity
7. pre-treating by stirring at 100-5,000 rpm at 30-70° C. for 1-3 h
8. sterilizing by filtration at the same pre-treatment temperature at a pressure of 1.5-6 bar
9. packaging the final filtered product in vials, up to the desired volume and closing with a butyl elastomer cap with aluminum ring, obtaining a product similar to the products shown in FIGS. 4 and 5
10. carrying out the necessary tests to certify the sterility of the product.

1. A process for the preparation of sterile products, comprising in sequence the phases of:
i. providing an aqueous solution comprising a thermolabile and/or bioactive raw material,
ii. pre-treating the aqueous solution maintaining stirring for 1-6 hours at 40-70° C., at a speed of 100-5.000 rpm;
iii. filtering the aqueous solution through at least one filter, at the same temperature of the pre-treatment phase ii), wherein the flow of the aqueous solution through the at least one filter defines an external surface (a) of the filter through which the aqueous solution enters into the filter and an internal surface (b) of the filter through which the sterile product exits;
wherein the at least one filter is a pleated filter comprising at least one layer consisting of a filtering membrane of polyethersulfone with pores having a nominal diameter smaller than or equal to 0.25 μm.

2. The process of claim 1, wherein the pleated filter is a multilayer filter, comprising at least three layers, wherein the filtering membrane of polyethersulfone forms a central layer in a sandwich between two further layers, consisting of an external support membrane and an internal support membrane, adapted to space the pleats of the filter apart by 0.1 mm.

3. The process of claim 2, wherein the pleated filter further comprises at least one or at least two external stiff lattices placed more externally with respect to the external support membrane, and the at least one or at least two internal stiff lattices placed more internally with respect to the internal support membrane, the internal or external stiff lattices being adapted to space the pleats of the filter apart.

4. The process of claim 1, wherein the thermolabile and/or bioactive raw material is hyaluronic acid or a derivative thereof, having a molecular weight of from 500 Da to 3 MDa and a concentration by weight in the aqueous solution of 0.05-10% based on the total weight of the aqueous solution.

5. The process of claim 1, wherein phase ii) of pre-treating the aqueous solution is carried out at a temperature of 50-70° C.

6. The process of claim 1, wherein phase iii) of filtering is carried out at a pressure of 1-8 bar.

7. The process of claim 1, wherein the phase i) of providing the aqueous solution comprises in sequence:
i.a) mixing in water hydrosoluble compounds of the aqueous solution, obtaining a first solution (A);
i.b) mixing preservatives and/or compounds that are soluble in glycol or glycerin, obtaining a second solution (B) and adding the second solution B to the first solution A, obtaining a third solution (C);
i.c) adding the thermolabile and/or bioactive raw material to the third solution C if present, or to the first solution A, obtaining the aqueous solution comprising the thermolabile and/or bioactive raw material of phase i);
wherein the sub-phases i.a)-i.e) are carried out at room temperature.

8. A closed circuit plant for carrying out the process of claim 1, the plant comprising a filtration system comprising at least one of the pleated filter, for filtering the aqueous solution, wherein the at least one filter is connected to at least one mixing tank with a thermostatic jacket upstream and to a filling and capping machine downstream, the at least one mixing tank being configured to perform pre-treatment of the aqueous solution.

9. Cosmetic use of a sterile product obtainable by the process of claim 1 in cosmetic mesotherapy or in aesthetic medicine, the sterile product being preferably in injectable form.

10. A sterile product obtainable by the process of claim 1 for use in dermatology in injectable form.
